(19) United States
(12) Patent Application Publication
(10) Pub. No.: US 2026/0031236 A1
(43) Pub. Date: Jan. 29, 2026

(54) TELEHEALTH SYSTEM AND METHOD FOR EMERGENCY RISK DETECTION

(71) Applicant: Jérémie NEUBERG, Paris (FR)

(72) Inventor: Jérémie NEUBERG, Paris (FR)

(21) Appl. No.: 19/347,822

(22) Filed: Oct. 2, 2025

Related U.S. Application Data
(63) Continuation-in-part of application No. 17/611,733.

(30) Foreign Application Priority Data
May 16, 2019 (FR) 1905164

(51) Int. Cl. G16H 50/30; G08B 21/02; G08B 21/18; G08B 25/00; G16H 40/67; G16H 80/00

(52) U.S. Cl. CPC G16H 50/30; G08B 21/02; G08B 21/182; G08B 25/001; G08B 25/005; G16H 40/67; G16H 80/00

(57) ABSTRACT

A telehealth system configured for remote monitoring and emergency response. The system includes at least one peripheral device for acquiring at least two physiological signals and at least one user-related information, a central server, and a plurality of recipient devices. A processor combines the acquired signals and information into a contextualized risk index, which is evaluated against multiple families of reference data, including general physiological thresholds, user-specific baselines, and instrument-specific reliability indicators. Based on this evaluation, the system determines whether a critical medical danger situation is occurring. When such a situation is detected, the system automatically outputs an emergency message to at least one recipient device selected from the plurality of recipient devices, thereby triggering timely intervention and facilitating rescue of the user.

### FIELD

The present invention pertains to the field of telehealth. More precisely it relates to a telehealth system and associated method for detecting and evaluating medical risk situations and for initiating emergency alerting rescuing when a critical medical danger situation is identified.

### BACKGROUND

Remote health monitoring and telehealth systems have been widely explored in the prior art. Existing platforms, such as those described in WO2016126859 and WO2017007461, generally focus on aggregating medical records or supporting automated decision-making through parsing and processing patient data. While such systems facilitate access to medical information and may support healthcare workflows, they are not designed to address real-time situations occurring outside controlled environments.

Conventional remote monitoring devices, including wearable sensors and hospital telehealth infrastructures, are generally designed to collect and transmit physiological data (e.g., heart rate, blood pressure, or oxygen saturation). While such systems can support clinical diagnosis and long-term patient management, they typically rely on either patient input or acknowledgment, or centralized medical review, before issuing an emergency alert. This reliance introduces latency and creates a high risk of failure in cases where the patient becomes incapacitated during acute crises such as cardiac arrest, respiratory collapse, or anaphylaxis.

Another limitation of prior art systems is their lack of robustness in heterogeneous personal-device networks. In practical environments, connected devices may differ in communication protocol, connectivity quality, and availability. Current systems do not adequately address the resulting challenges of alert delivery latency and transmission failure rates across such diverse communication infrastructures. As a result, emergency alerts may not reliably reach assistance providers within the critical first seconds following detection of a medical event, thereby reducing survival chances.

The core technical problem addressed by the present invention is that existing remote health monitoring systems do not provide automatic and reliable detection of imminent medical danger, nor do they ensure low-latency and reliable delivery of alerts across heterogeneous device networks, particularly when the patient is incapacitated and unable to intervene.

An objective of the present invention is therefore to provide a telehealth system and method that overcome these limitations by performing multi-level evaluation of physiological and user-related data, integrating user-specific baselines and signal reliability indicators, and initiating prioritized low-latency emergency alerting to appropriate assistance providers or professional services, without requiring patient intervention.

### SUMMARY

The present invention relates to a telehealth system for rescuing a user in a critical medical danger situation, the telehealth system comprising a central server, a plurality of recipient devices, and at least one peripheral device of the user;
the peripheral device and plurality of recipient devices being communicatively coupled to the central server;
the peripheral device being configured to continuously or intermittently obtain at least two physiological signals indicative of a health condition of said user, and at least one user-related information;
wherein said telehealth system comprises:
   at least one input configured to receive:
      said at least two physiological signals and said at least one user-related information;
      a list of the plurality of recipient devices;
      a plurality of reference data families, including physiological reference data, user-specific reference data, and instrument-specific reference data;
   at least one processor configured to:
      combine the received at least two physiological signals and user-related information into a contextualized risk index;
      evaluate the contextualized risk index against the plurality of reference data families, wherein the evaluation comprises a multi-level comparison including:
         comparing the contextualized risk index to said general physiological reference data representing population-based medically established safety thresholds, wherein a breach of said general physiological reference data automatically indicates a critical medical danger situation regardless of other reference data families; and
         refining the evaluation of the contextualized risk index using user-specific reference values representing individualized baselines, and instrument-specific reference values indicative of a reliability level of the acquired signals;
      determine, on the basis of said evaluation, whether said contextualized risk index indicates that said critical medical danger situation is occurring;
   at least one output configured to, when said critical medical danger situation is occurring, send an emergency message to at least one recipient device selected from the list of said plurality of recipient devices, thereby triggering an intervention to rescue said user in response to the detected critical medical danger situation.

Advantageously, the telehealth system enables rapid and accurate detection of critical medical danger situations by combining multi-modal physiological monitoring with reliability-aware signal processing and automated emergency communication. To this end, multiple physiological signals and user-related information are combined into a contextualized risk index, with the evaluation of said index further informed by a plurality of reference data families. This approach ensures that only truly dangerous events trigger emergency messages, thereby minimizing false alarms and avoiding unnecessary strain on emergency services. By incorporating general physiological reference data, user-specific reference values, and instrument-specific reference values, the system achieves a balance between population-level safety thresholds, individualized baselines, and device reliability. This allows to more precisely identify critical medical danger situations, enhancing both sensitivity and specificity. Furthermore, the telehealth system allows timely intervention by automatically transmitting emergency messages based on detected signals, even in situations where the user is unaware of the danger or is unconscious, ensuring rapid response when it is most critical. The multi-level evaluation also reduces unnecessary data transmission, conserving bandwidth and minimizing latency, which collectively ensures a fast, resource-efficient, and reliable response in critical situations.

According to an embodiment, the at least two physiological signals are selected from different categories of vital parameters, including but not limited to cardiac (e.g., ECG, heart rate, heart rate variability), respiratory (e.g., respiration rate, SpO₂, airflow), circulatory (e.g., blood pressure, perfusion index), and metabolic (e.g., glucose, temperature) signals. The at least one user-related parameter includes for instance demographic characteristics, medical history, medication regimen, or activity context.

According to an embodiment, the contextualized risk index integrates the physiological signals and user-related parameters using a dynamically weighted combination in which weighting factors are adapted in real time. For instance, weighting factors are adapted according to instrument-specific reliability data received as input.

According to other advantageous aspects of the invention, the device for obtaining at least one trained machine learning model comprises one or more of the features described in the following embodiments, taken alone or in any possible combination.

According to an embodiment, the evaluation of the contextualized risk index is performed over at least one sliding temporal window of configurable duration, and wherein a deviation of the contextualized risk index from at least one of said reference data families is considered for the evaluation of the contextualized risk index only if the deviation persists for a minimum duration within said sliding temporal window.

By requiring that a deviation of the contextualized risk index from one or more reference families persists for a minimum duration within a sliding temporal window before it is taken into account, the telehealth system becomes significantly more robust against sensor noise, motion artifacts, temporary physiological variations and sustained deviations in the input data that are more indicative of genuine medical danger situations. In practice, physiological signals and user-related information are often affected by brief irregularities, such as movement, sensor misplacement, or environmental interference, which can create false alarms if interpreted at face value. By filtering out short-lived anomalies while still ensuring timely detection of persistent risk patterns, the invention reduces false positives and prevents unnecessary emergency interventions, thereby improving both the reliability and the efficiency of real-time telehealth rescue systems. The use of a sliding temporal window ensures that the evaluation is continuous and adaptive, rather than tied to fixed intervals, thereby improving both responsiveness and accuracy. The configurable duration parameter further allows customization depending on the medical context. For example, a shorter window can be implemented for acute cardiac monitoring where rapid changes are critical, while a longer window may be implemented for chronic conditions where stability is more important. From a safety perspective, this mechanism filters out noise while preserving true critical events, meaning that the system reacts to conditions that are clinically relevant rather than incidental.

According to an embodiment, the evaluation of the contextualized risk index further comprises applying a machine learning model configured to receive as input the physiological signals and the user-related parameters and to output a refined evaluation of the contextualized risk index, wherein the machine learning output does not override a deviation of the contextualized risk index from said general physiological reference data.

The output may be expressed for example as a probability value or as a categorical severity level of a critical medical danger situation. In addition, or alternatively, the machine learning model may dynamically adjust the weighting factors used in the computation of the contextualized risk index, such that inputs with higher reliability or stronger correlation to the user's condition exert greater influence on the contextualized risk index.

The machine learning contribution is used to complement and refine the evaluation of the contextualized risk index but remains subject to a safety hierarchy in which any deviation of the contextualized risk index from the general physiological reference data is decisive and cannot be overridden. This safeguard is enforced at the system logic level to ensure that breaches of medically established population thresholds always trigger recognition of a critical medical danger situation, regardless of machine learning output. By combining adaptive and individualized refinement with absolute safety guarantees, the system provides a robust technical solution that improves sensitivity and specificity while preventing under-alerting due to model error, bias, or adversarial input. These two modes of operation (i.e., direct output of a refined risk evaluation and indirect adjustment of weighting factors in the contextualized risk index computation) are alternative or complementary implementations of the same machine learning module.

By introducing the use of a machine learning model for evaluating the physiological signals and user-related information, the invention gains an additional layer of adaptability and predictive power. Conventional telehealth systems typically rely on rigid, rule-based thresholds, whereas a trained model can identify subtle correlations between multiple physiological signals and user-related information that might precede a critical medical danger situation. This allows the system to detect risks earlier and with greater nuance than a purely deterministic ruleset. At the same time, the telehealth system ensures that the machine learning output does not override the hard safety thresholds defined by the general physiological reference values. This is a critical point: it maintains compliance with established medical safety standards, thereby preventing the system from ever ignoring a clearly dangerous situation solely on the basis of the machine learning model output. This safeguard not only reinforces patient safety but also avoids regulatory concerns that could otherwise arise from reliance on a “black box” model.

According to another embodiment, the evaluation of the contextualized risk index further comprises applying a machine learning model configured to receive, as inputs, the contextualized risk index, optionally together with underlying features received as input and/or derived from the physiological signals and user-related information received as input, said underlying features including user baseline values, temporal signal patterns, and sensor reliability indicators, and to output a refined evaluation of the contextualized risk index.

This refined evaluation of the contextualized risk index can take the form of a probability score, severity classification, or adjusted risk index. The refined evaluation of the contextualized risk index may be combined with the evaluation against the user-specific and instrument-specific reference data families to increase robustness and reduce false positives, while the evaluation against general physiological reference data remains decisive such that any breach of said physiological reference data automatically indicates a critical medical danger situation regardless of the machine learning output.

According to an embodiment, the determination whether said critical medical danger situation is occurring comprises subdividing the critical medical danger situation into at least two levels of criticality, each level of criticality corresponding to a different severity of deviation of the contextualized risk index. The subdivision may be performed within the same multi-level evaluation framework that integrates population thresholds, user-specific baselines, and instrument-specific reliability values. Each level of criticality is associated with a differentiated response protocol, enabling graduated escalation of alerts according to a severity of the detected risk.

By deriving criticality levels from the contextualized risk index rather than from raw physiological thresholds alone, the system provides a structured evaluation framework that improves triage efficiency, reduces unnecessary emergency activations, and ensures that sustained or severe deviations are prioritized for immediate intervention. This approach distinguishes the invention from prior art systems relying on single-threshold alarms, as it introduces a hierarchical classification mechanism specifically adapted to multi-modal and reliability-weighted telehealth monitoring.

According to an embodiment, the plurality of recipient devices comprises at least one potential assistance provider recipient device and at least one professional emergency recipient device.

According to the invention, potential assistance provider recipient devices correspond to devices of non-professional individuals located within a defined geographical or network-based proximity to the user, while professional emergency recipient devices correspond to devices or systems operated by formal emergency response services.

This structured recipient categorization, in combination with the multi-level evaluation of the contextualized risk index, provides an optimized rescue initiation that minimizes time of first response, while preventing overloading of emergency services with non-critical alerts.

From a practical perspective, this arrangement allows faster preliminary intervention: immediate contacts who are familiar with the user can be notified right away, potentially stabilizing the user or providing guidance until professional emergency services arrive. This dual-recipient framework improves overall responsiveness and patient outcomes, reducing the risk of serious harm in the interval before professional care is available.

Such integration of differentiated recipient classes into a risk-index-driven escalation framework is not taught in prior art systems, which typically rely on flat distribution of alerts to a single class of recipients.

According to an embodiment, said at least two levels of criticality include at least one lower level and at least one higher level, wherein in the at least one lower level, the at least one output is configured to prioritize sending said emergency message to said potential assistance provider recipient device, and in the at least one higher level, the at least one output is configured to prioritize sending said emergency message to said professional emergency recipient device.

In other words, the telehealth system is configured to associate the different categories of recipient devices with different levels of criticality of the contextualized risk index, such that lower levels of criticality trigger alerts primarily to potential assistance providers to ensure rapid preliminary intervention, while higher levels of criticality trigger alerts directly to professional emergency services, either alone or in parallel with assistance providers.

The prioritization logic may comprise sequential transmission, parallel transmission with differentiated weighting, or escalation within a predetermined time interval if confirmation of assistance is not received.

By linking the escalation pathway directly to the contextualized, multi-level evaluation framework, the system ensures that recipient selection is not based on static or manual rules, but on an automated, reliability-weighted assessment of the user's real-time condition. This integration reduces false alarms to professional emergency services, minimizing unnecessary dispatches, and simultaneously accelerating the first available intervention. Unlike prior art systems that implement fixed escalation chains independent of the quality or persistence of physiological deviations, the present invention couples escalation logic to a dynamically computed risk index, thereby achieving an adaptive and technically robust telehealth rescue mechanism.

According to an embodiment, said at least one output is configured to send an emergency message to at least one potential assistance provider recipient device selected from the list of the plurality of recipient devices, wherein the selection is based on a multi-factor evaluation that combines: (i) a physical proximity of the potential assistance provider recipient device (e.g., determined from GPS, Bluetooth) to the user or a network-based proximity to the user, (ii) a technical availability of the potential assistance provider recipient device, and (iii) a medical or relational relevance of a potential assistance provider associated with the potential assistance provider recipient device. The technical availability of the potential assistance provider recipient device may be determined from its network connectivity status, battery level, or readiness to receive alerts. The medical or relational relevance of the potential assistance provider associated with said device may be determined from stored role information (such as trained first responder, healthcare worker, or designated relative) or from prior authorization settings within the system.

In one embodiment, the telehealth system evaluates the proximity of each potential assistance provider recipient device using both physical and network criteria. Physical proximity refers to the geographical closeness of the device to the user, while network proximity refers to the communication latency or signal availability between the device and the central server. By combining these two parameters, the system selects at least one recipient device that can acknowledge and receive the alert with the lowest expected delay. This embodiment illustrates a narrower case of the more general multi-factor selection described in later embodiments.

By applying this combined evaluation, the system automatically prioritizes the recipient device most capable of delivering timely and effective assistance. This multi-factor selection mechanism maximizes response reliability and minimizes unnecessary or misdirected alerts. Compared to prior art systems that rely only on proximity or static recipient lists, the present invention achieves improved responsiveness, reduced alert failure, and greater robustness in heterogeneous device networks.

According to an embodiment, said at least one output is configured to send an emergency message to at least one potential assistance provider recipient device selected from the list of the plurality of recipient devices, wherein the selection is based on an evaluation of a physical proximity of each potential assistance provider recipient device to prioritize a potential assistance provider recipient device that is closest to the user. The system prioritizes the potential assistance provider recipient device that is geographically closest to the user, as determined for example from geo-localization data obtained via GPS, Bluetooth signal strength, Wi-Fi association, or other location-based indicators. By using physical proximity as the decisive criterion, the system ensures that the emergency message is directed first to a recipient who is most likely able to reach the user within the shortest time frame, thereby increasing the likelihood of immediate intervention in critical medical danger situations.

According to an embodiment, said at least one output is configured to send an emergency message to at least one potential assistance provider recipient device selected from the list of the plurality of recipient devices, wherein the selection is based on an evaluation of a network proximity of each potential assistance provider recipient device to prioritize a potential assistance provider recipient device that is capable of receiving the emergency message with a lowest communication latency.

According to the invention, the network proximity of the recipient device is defined as the expected latency of delivering the emergency message, determined for example from real-time measurements of network response times or signal quality.

By evaluating the physical or network proximity of each potential assistance provider recipient device, the system can select the device most capable of delivering timely aid, either because the associated human responder is physically closest to the user or because the device is reachable fastest over the network. This ensures that in critical medical danger situations, intervention can occur as quickly as possible, potentially stabilizing the user before professional emergency services arrive. It also optimizes system resources by avoiding alerts to devices that are distant or likely unreachable, reducing unnecessary data transmission and minimizing delays.

The emergency message is directed to the recipient device that satisfies both physical proximity and technical availability. The combined evaluation is performed within the contextualized, multi-level risk index framework, such that only recipient devices that are geographically close to the user and demonstrably available to receive alerts are prioritized. This embodiment ensures that emergency messages are delivered to the recipient most capable of responding quickly and reliably, thereby reducing the risk of missed or delayed interventions compared to systems relying on a single selection criterion.

According to an embodiment, said at least one output is configured to send an emergency message to at least one potential assistance provider recipient device selected from the list of the plurality of recipient devices, wherein the selection is based on an evaluation of a technical availability status of each potential assistance provider recipient device, said technical availability status comprising at least one of: a battery level, a connectivity quality, a device activity state, and a user interaction state.

According to an embodiment, said at least one output is configured to send an emergency message to at least one potential assistance provider recipient device selected from the list of the plurality of recipient devices, wherein the selection is based on an evaluation of a medical or relational relevance of the potential assistance provider associated with said potential assistance provider recipient device, said medical or relational relevance being defined according to at least one of: a professional qualification of the potential assistance provider, a pre-registered emergency contact designation, and a degree of relational proximity with the user.

According to an embodiment, said at least one output is configured to send an emergency message to at least one potential assistance provider recipient device selected from the list of the plurality of recipient devices, wherein the selection is based on a combination of a physical and/or network proximity score, a technical availability score, and a medical or relational relevance score into a weighted prioritization function, wherein a highest-ranked potential assistance provider recipient device is selected to receive the emergency message.

According to an embodiment, if the selected potential assistance provider recipient device does not acknowledge receipt of the emergency message within a predetermined response time, the at least one output is configured to send the emergency message to an alternative potential assistance provider recipient device selected from the list of the plurality of recipient devices.

The acknowledgment may comprise (i) a device-level confirmation, such as an automatic system log indicating successful message delivery to the recipient device; or (ii) a user-level confirmation, such as a manual input, acceptance, or interaction by the human recipient. By distinguishing between automatic and manual confirmation modes, the system ensures robust detection of whether an emergency message has been effectively received and acted upon.

This mechanism increases the robustness of the emergency communication process by ensuring that an alternative recipient is automatically contacted if the initially selected device is unavailable, thereby reducing the risk of failed or delayed rescue initiation.

According to an embodiment, the alternative potential assistance provider recipient device is selected based on the same combination of three factors used to select the selected potential assistance provider recipient device, including a proximity of the alternative potential assistance provider recipient device to the user, (ii) a technical availability of the alternative potential assistance provider recipient device, and (iii) a medical or relational relevance of a potential alternative assistance provider associated with the alternative potential assistance provider recipient device.

According to an embodiment, successive alternative recipient devices are sequentially notified until at least one potential assistance provider recipient device acknowledges the emergency message.

Successive alternative recipient devices may be automatically notified in a predetermined or dynamically updated sequence until at least one potential assistance provider recipient device acknowledges the emergency message. The sequence may be established using the same prioritization criteria applied for the initial selection, including physical or network proximity, technical availability, and medical or relational relevance, thereby ensuring that the most suitable alternative recipients are contacted first.

This sequential notification mechanism increases the probability that at least one recipient device will respond in time to initiate assistance, while avoiding unnecessary broadcasting of the emergency message to all recipients at once, thereby optimizing communication resources and reducing alert fatigue.

According to an embodiment, the alternative recipient device corresponds to the second-ranked potential assistance provider recipient device.

According to an embodiment, said at least one output is configured to escalate the emergency message to said at least one professional emergency recipient device if all potential assistance provider recipient devices fail to respond within a predetermined duration.

The escalation trigger may be based on the absence of device-level confirmation of receipt or user-level confirmation of acceptance, and is enforced automatically by the system logic without requiring user intervention. This escalation mechanism ensures that a critical medical danger situation is always addressed, providing a fail-safe path to professional emergency services when local assistance providers are unavailable or unresponsive.

According to an embodiment, said at least one output is configured to automatically escalate the emergency message to said at least one professional emergency recipient device if no acknowledgment of a potential assistance provider recipient device is recorded within a predetermined interval of time At. At is a system parameter stored in the telehealth system logic and is dynamically adjustable according to the level of criticality of the contextualized risk index. For higher levels of criticality, At is configured to be shorter in order to guarantee immediate escalation, whereas for lower levels of criticality, At may be set longer to allow local responders an opportunity to intervene. This escalation mechanism provides a fail-safe pathway to professional emergency services by ensuring that, regardless of local response availability, a critical medical danger situation will always be addressed within a defined maximum delay.

According to an embodiment, said at least one output is further configured to escalate the emergency message to professional emergency recipient devices upon a change in the level of criticality of the critical medical danger situation after an initial emergency message has been sent to at least one of said potential assistance provider recipient devices.

The system continuously or intermittently re-evaluates the contextualized risk index based on incoming physiological signals, and any transition from a lower to a higher level of criticality automatically triggers escalation to professional emergency services. This mechanism provides immediate escalation to professional services when the user's condition worsens, ensuring rapid intervention in evolving emergencies.

[The following claims are from the first patent application. They are added here to keep basis for claiming priority of the first patent application]

According to one embodiment, the emergency message may comprise a user's identification data and geo-localization data, such as for example geo-localization data obtained via user's authorizing, via Bluetooth, via triangulation, via WIFI network.

This embodiment ensures that responders, whether potential assistance providers or professional emergency personnel, are able to quickly determine who requires help, which is crucial in environments where multiple users may be monitored or in cases where the user is unable to communicate.

According to one embodiment, the proximity factor comprises being connected to the same wireless network as the user's peripheral device.

This embodiment allows to use network proximity as an indicator of physical or functional closeness between the user and potential assistance provider recipient devices. By prioritizing devices that are connected to the same wireless network as the user's peripheral device, the system can identify recipients who are likely physically nearby or within immediate reach, without requiring explicit location tracking. This approach improves the speed and effectiveness of emergency response, since notifications are sent first to responders who are most likely to be able to act quickly. It also enhances system efficiency, as it avoids sending alerts to devices that are distant or network-disconnected, thereby conserving bandwidth and reducing the likelihood of missed or delayed notifications.

According to one embodiment, the proximity factor comprises being located within a predetermined distance ranging between 0 meters and 500 meters. In an embodiment, the predetermined distance is ranging between 1 meter and 100 meters.

According to one embodiment, the user-related information comprises:
an electronic medical record of the user and/or
the user's answers to the questions of a questionnaire, the questionnaire comprising questions that are neutral to the user and questions that are generated by means of an algorithm based on the at least two physiological signals;
   and wherein the determination of the contextualized risk index is further based on the data comprised in the electronic medical record and/or the user's answers to the questions of the questionnaire.

According to one embodiment, the electronic medical record comprises genetic factors and/or epidemiologic factors.

According to one embodiment, the critical medical danger situation is selected from a group comprising: anaphylactic shock, stroke, heart failure, cardiovascular diseases, respiratory deficiency, sleep apnea, endocrinal disease, metabolic disease, infectious or parasitic diseases, digestive diseases, immune system diseases, nephrological disorders, sexually transmitted diseases, viral diseases, smoking addiction, drug use addiction, arterial hypertension, cancer, kidney diseases, gynecological diseases, neurodegenerative diseases, neurological diseases, psychiatric diseases, post-operative risk, circulatory system disease, sudden cardiac death, nutritional diseases or a risk of sudden death.

According to one embodiment, said at least two physiological signals are selected from a group comprising: oxygen saturation, respiratory rate, blood pressure, heart rate, temperature, weight, respiratory rate, blood oxygen saturation, pulse rate, heart rate variability, stroke volume, cardiac output, cardiac index, pulse pressure, systemic vascular resistance, ECG, mean arterial pressure, sweat level, skin temperature, body temperature, glucose level.

According to one embodiment, the predetermined interval of time At ranges between 10 and 60 seconds. The predetermined interval of time At for a moderate risk index level is superior to the interval of time At for a high risk index level.

According to one embodiment, the emergency message further comprises:
a questionnaire;
a medicine prescription;
a medicine purchase order for the prescribed medicine;
automatically delivering the prescribed medicine;
a pre-medical evaluation report;
a post-medical evaluation report;
a user's consultation with a healthcare provider;
sending a medication reminder to the user;
sending an alert to a healthcare provider.

[End of claims from the first patent application]

The present invention also relates to a computer-implemented method for rescuing a user in a critical medical danger situation, the method being implemented by a telehealth system comprising a central server, a plurality of recipient devices, and at least one peripheral device of the user;
the peripheral device and plurality of recipient devices being communicatively coupled to the central server;
the peripheral device being configured to continuously or intermittently obtain at least two physiological signals indicative of a health condition of said user, and at least one user-related information;
wherein said method comprises:
   receiving:
      said at least two physiological signals and said at least one user-related information;
      a list of the plurality of recipient devices;
      a plurality of reference data families, including physiological reference data, user-specific-reference data, and instrument-specific reference data;
   combining the received at least two physiological signals and user-related information into a contextualized risk index;
   evaluating the contextualized risk index against the plurality of reference data families, wherein the evaluation comprises a multi-level comparison including:
      comparing the contextualized risk index to said general physiological reference data representing population-based medically established safety thresholds, wherein a breach of said general physiological reference data automatically indicates a critical medical danger situation regardless of other reference families; and
      refining the evaluation of the contextualized risk index using user-specific reference values representing individualized baselines, and instrument-specific reference values indicative of a reliability level of the acquired signals;
   determining, on the basis of said evaluation, whether said contextualized risk index indicates that the critical medical danger situation is occurring;
   automatically outputting, when said critical medical danger situation is occurring, an emergency message to at least one recipient device selected from the list of the plurality of recipient devices, thereby triggering an intervention to rescue said user in response to the detected critical medical danger situation.

In one embodiment, the determination of whether said contextualized risk index indicates that the critical medical danger situation is occurring is carried out in real time.

In one embodiment, the at least one recipient device is selected from the list of the plurality of recipient devices based on contextualized prioritization criteria.

In one embodiment, the method of the present invention further comprises a step b.2) of evaluating a quality metrics of the at least two physiological signals and said at least one user-related information by means of an artifact detection algorithm;
and the step f) of evaluating the contextualized risk index is further based on the evaluated quality metrics.

In one embodiment, the list of the plurality of recipient devices is generated using the following steps:
receiving as input information regarding at least peripheral device of the user and at least one recipient device designated by the user;
identifying neighbor devices based on a user's vicinity criterion; the preferred vicinity criterion being: identifying the recipient devices that are connected to the same wireless network as the at least one user's peripheral device.
automatically generating the list of the plurality of recipient devices comprising, as recipients: the at least one user's peripheral device; the at least one recipient device designated by the user and the identified neighbor devices.

In one embodiment of the present, the method further comprises:
obtaining at least two physiological signals and said at least one user-related information from a plurality of users;
building, by means of a machine learning algorithm, a predictive model based on the data from a plurality of users.

In one embodiment, the present method further comprises a step of inputting the at least two physiological signals and said at least one user-related information into the predictive model and obtaining as output a value predictive of a future clinical event of the user.

In addition, the disclosure relates to a computer program comprising software code adapted to perform the computer-implemented method for rescuing a user in a critical medical danger situation compliant with any of the above execution modes when the program is executed by a processor.

The present disclosure further pertains to a computer-readable medium comprising instructions which, when executed by a computer, cause the computer to carry out the computer-implemented method for rescuing a user in a critical medical danger situation compliant with any of the above execution modes.

The present disclosure further pertains to a non-transitory program storage device, readable by a computer, tangibly embodying a program of instructions executable by the computer to perform the computer-implemented method for rescuing a user in a critical medical danger situation compliant with the present disclosure.

Such a non-transitory program storage device can be, without limitation, an electronic, magnetic, optical, electromagnetic, infrared, or semiconductor device, or any suitable combination of the foregoing. It is to be appreciated that the following, while providing more specific examples, is merely an illustrative and not exhaustive listing as readily appreciated by one of ordinary skill in the art: a portable computer diskette, a hard disk, a ROM, an EPROM (Erasable Programmable ROM) or a Flash memory, a portable CD-ROM (Compact-Disc ROM).

### Definitions

In the present invention, the following terms have the following meanings:

The terms “adapted” and “configured” are used in the present disclosure as broadly encompassing initial configuration, later adaptation or complementation of the present device, or any combination thereof alike, whether effected through material or software means (including firmware).

The term “processor” should not be construed to be restricted to hardware capable of executing software, and refers in a general way to a processing device, which can for example include a computer, a microprocessor, an integrated circuit, or a programmable logic device (PLD). The processor may also encompass one or more Graphics Processing Units (GPU), whether exploited for computer graphics and image processing or other functions. Additionally, the instructions and/or data enabling to perform associated and/or resulting functionalities may be stored on any processor-readable medium such as, e.g., an integrated circuit, a hard disk, a CD (Compact Disc), an optical disc such as a DVD (Digital Versatile Disc), a RAM (Random-Access Memory) or a ROM (Read-Only Memory). Instructions may be notably stored in hardware, software, firmware or in any combination thereof.

“Machine learning (ML)” designates a class of computer algorithms that automatically improve their performance on a defined task through experience. ML models are trained on datasets comprising input features and corresponding outputs, enabling the models to iteratively adjust internal parameters so as to minimize the difference between predicted and expected results. This process allows the system to identify complex correlations, patterns, or predictive indicators in multi-modal physiological and user-related data, thereby enhancing adaptability and accuracy in evaluating medical risk situations. In the context of the present invention, ML outputs are expressly designed to complement, but not override, medically established safety thresholds derived from general physiological reference data, ensuring that patient safety remains decisive regardless of model behavior.

“Datasets” are collections of data used to build an ML mathematical model, so as to make data-driven predictions or decisions. In “supervised learning” (i.e. inferring functions from known input-output examples in the form of labelled training data), three types of ML datasets (also designated as ML sets) are typically dedicated to three respective kinds of tasks: “training”, i.e. fitting the parameters, “validation”, i.e. tuning ML hyperparameters (which are parameters used to control the learning process), and “testing”, i.e. checking independently of a training dataset exploited for building a mathematical model that the latter model provides satisfying results.

A “neural network (NN)” designates a category of ML comprising nodes (called “neurons”), and connections between neurons modeled by “weights”. For each neuron, an output is given in function of an input or a set of inputs by an “activation function”. Neurons are generally organized into multiple “layers”, so that neurons of one layer connect only to neurons of the immediately preceding and immediately following layers.

The above ML definitions are compliant with their usual meaning, and can be completed with numerous associated features and properties, and definitions of related numerical objects, well known to a person skilled in the ML field. Additional terms will be defined, specified or commented wherever useful throughout the following description.

A “critical medical danger situation” refers to any physiological or health-related condition of the user that poses an immediate or imminent risk to the user's well-being or life. Such a situation is specifically medical in nature and is independent of external environmental hazards, accidents, or other non-medical emergencies. Examples of critical medical danger situations include, without limitation, acute cardiac events (e.g., arrhythmias, myocardial infarction), respiratory crises (e.g., severe hypoxemia, apnea), sudden drops or spikes in blood pressure, severe hypoglycemia or hyperglycemia, and life-threatening deviations in core body temperature or other vital signs.

### BRIEF DESCRIPTION OF THE DRAWINGS

The present disclosure will be better understood, and other specific features and advantages will emerge upon reading the following description of particular and non-restrictive illustrative embodiments, the description making reference to the annexed drawings wherein:

FIG. 1 is a block diagram representing schematically a telehealth system compliant with the present disclosure;

FIG. 2 is a block diagram representing schematically a particular mode of a telehealth system, compliant with the present disclosure;

FIG. 3 is a flow chart showing successive steps of the computer-implemented method executed by the telehealth system of FIG. 2;

FIG. 4 diagrammatically shows an apparatus integrating the functions of the devices of FIGS. 1 to 3;

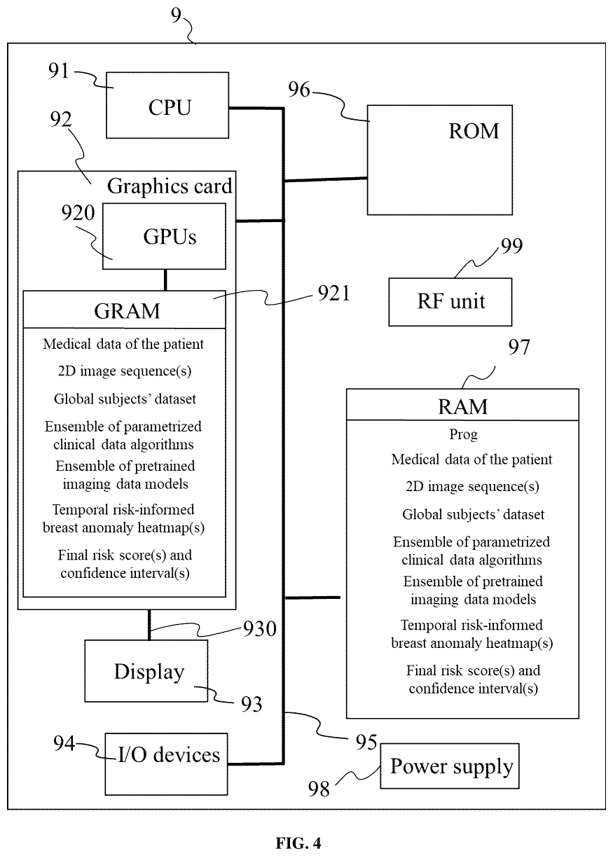

FIG. 5 is a flowchart illustrating features of the telehealth system of FIG. 2, according to a first embodiment; and

FIG. 6 is a flowchart illustrating features of the telehealth system of FIG. 2, according to a second embodiment.

On the figures, the drawings are not to scale, and identical or similar elements are designated by the same references.

### DETAILED DESCRIPTION

The present description illustrates the principles of the present disclosure. It will thus be appreciated that those skilled in the art will be able to devise various arrangements that, although not explicitly described or shown herein, embody the principles of the disclosure and are included within its scope.

All examples and conditional language recited herein are provided to aid in understanding the principles of the disclosure and the concepts contributed by the inventor to furthering the art, and are to be construed as being without limitation to such specifically recited examples and conditions.

Moreover, all statements herein reciting principles, aspects, and embodiments of the disclosure, as well as specific examples thereof, are intended to encompass both structural and functional equivalents thereof. Additionally, it is intended that such equivalents include both currently known equivalents as well as equivalents developed in the future, i.e., elements that perform substantially the same function in substantially the same way, to achieve the same result, regardless of structure.

Thus, for example, it will be appreciated by those skilled in the art that the block diagrams presented herein may represent conceptual views of illustrative circuitry embodying the principles of the disclosure. Similarly, it will be appreciated that any flow charts, flow diagrams, and the like represent various processes which may be substantially represented in computer readable media and so executed by a computer or processor, whether or not such computer or processor is explicitly shown.

The functions of the various elements shown in the figures may be provided through the use of dedicated hardware as well as hardware capable of executing software in association with appropriate software. When provided by a processor, the functions may be provided by a single dedicated processor, a single shared processor, or a plurality of individual processors, some of which may be shared.

It should be understood that the elements shown in the figures may be implemented in various forms of hardware, software or combinations thereof. Preferably, these elements are implemented in a combination of hardware and software on one or more appropriately programmed general-purpose devices, which may include a processor, memory and input/output interfaces.

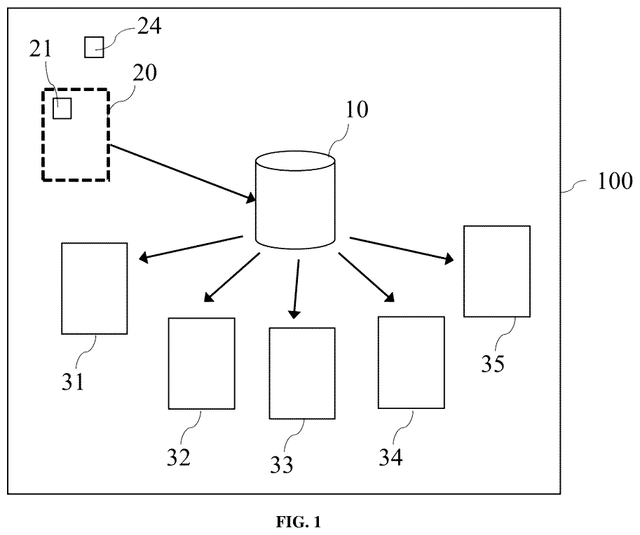

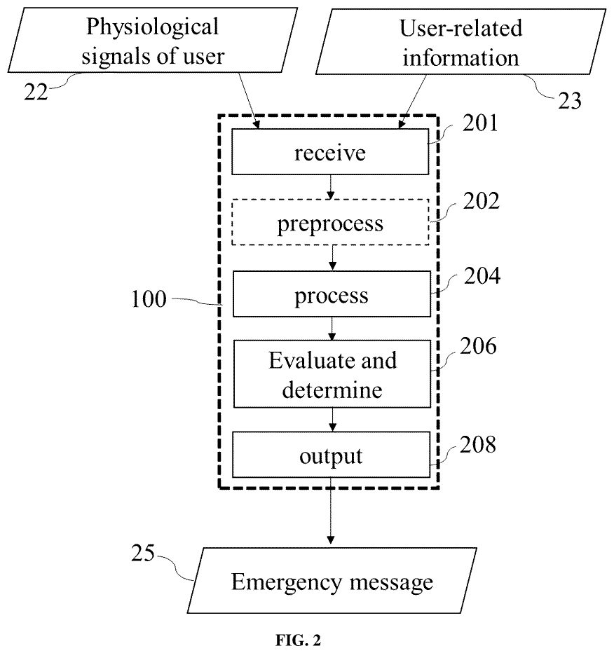

The present disclosure will be described in reference to a particular functional embodiment of a telehealth system 100 for rescuing a user in a critical medical danger situation as illustrated on FIG. 1 and FIG. 2.

The telehealth system 100 is more precisely suited for remote monitoring and emergency response for a user, including users with known medical conditions.

In certain embodiments, the architecture described herein yields technical effects that facilitate coordinated care: by computing a contextualized risk index from multi-modal signals and transmitting alerts with reduced latency, the system decreases manual triage load and shortens response times.

The system further enables automated storage of time-stamped physiological and user-related data in secure memory, supporting subsequent diagnostic review, adaptive baseline recalibration, and predictive model updates.

These technical effects indirectly support patient-centric workflows by enabling continuous monitoring and context-sensitive alerting without user intervention.

The aim of the present invention is to continuously improve and optimize the patient care pathways; to optimize the workflow of tasks performed by the healthcare professionals; to ease the communication between healthcare professionals; and to optimize the organization of healthcare facilities.

The present system maximizes the amount of a user's digital data that are available for diagnostic and therapeutic purpose; moreover, it automatizes the storage of said data with the twofold aim of improving the organization of healthcare facilities, such as hospitals and clinics, and implementing personalized and predictive medicine.

The purpose of the present invention is to deliver a patient-centered care, to ensure the best quality of care, to ensure that all the steps in the healthcare workflows fit together, especially the most critical steps that involve interactions between patients, healthcare professionals, general practitioners and healthcare facilities in order to meet the need of a community-based care.

The present invention is a telehealth system that allows the user play an active role throughout his/her care pathway.

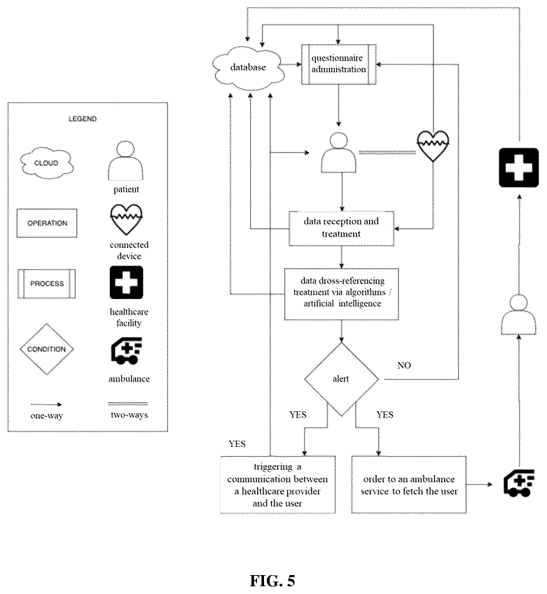

The system according to the present invention comprises a secure access to data, several algorithms and a plurality of connected medical devices, watches, patches, wearables, scales or other connected objects, which are capable of transmitting data of a user at a given moment of time or continuously. The combination of these elements allows the telehealth system to perform a plurality of functions, such as for example those illustrated in FIG. 5.

The telehealth system 100 is configured to receive at least two physiological signals 22 of a user and at least one user-related information 23 and to output at least one emergency message to trigger an intervention to rescue the user in response to the detected critical medical danger situation.

In one embodiment, the telehealth system 100 comprises a central server 10 communicatively coupled to at least one peripheral device 20 belonging to said user and to a plurality of recipient devices 30, each belonging to a potential assistance provider.

Though the presently described telehealth system 100 (i.e. comprising the central server 10, peripheral device 20 and recipient devices 30) is versatile and provided with several functions that can be carried out alternatively or in any cumulative way, other implementations within the scope of the present disclosure include devices having only parts of the present functionalities.

Each of the central server 10, peripheral devices 20 and recipient devices 30 is advantageously an apparatus, or a physical part of an apparatus, designed, configured and/or adapted for performing the mentioned functions and producing the mentioned effects or results. In alternative implementations, any of the central server 10, peripheral devices 20 and recipient devices 30 is embodied as a set of apparatus or physical parts of apparatus, whether grouped in a same machine or in different, possibly remote, machines. The central server 10, peripheral devices 20 and recipient devices 30 may e.g. have functions distributed over a cloud infrastructure and be available to users as a cloud-based service, or have remote functions accessible through an API.

The central server 10, recipient devices 30 and peripheral devices 20 may be integrated in a same apparatus or set of apparatus. In other implementations, the structure of the central server 10, peripheral device 20 and recipient devices 30 may be completely independent.

The peripheral device 20 is adapted for acquiring continuously or intermittently multiple physiological signals 22, wherein multiple physiological signals 22 includes at least two physiological signals 22. The peripheral device 20 is further configured for acquiring and/or receiving at least one user-related information 23 and to ensure transmission of said physiological signals 22 and user-related information 23, or data derived therefrom, to the central server 10 for further analysis and generation of appropriate assistance actions.

Notably, the peripheral device 20 is adapted for acquiring and/or receiving continuously or intermittently multiple user-related information 23, wherein multiple user-related information 23 includes at least two user-related information 23.

The peripheral device 20 may integrate at least one sensor 21, 24, directly within its structure (i.e. integrated sensors 21), and/or be associated with sensors that are external to the peripheral device 20 (i.e. external sensors 24), thereby enabling acquisition of user-related physiological signals 22 and user-related information 23.

In one embodiment, the peripheral device 20 may comprise at least one physiological sensor integrated in a wearable platform, such as a wristband, smartwatch, chest strap, adhesive patch, or a ring-type device. Examples include commercial devices such as Apple Watch®, Galaxy Watch®, or Fitbit®, capable of providing measurements of heart rate, SpO₂, heart rate variability (HRV), skin temperature, or respiratory rate.

In another embodiment, the peripheral device 20 may comprise an ear-worn sensor such as connected earbuds (e.g., Apple AirPods®, Galaxy Buds®) embedding optical or electrical sensors and microphones to detect physiological signals 22, in particular respiratory effort, breathing irregularities, or voice changes indicative of distress.

In another embodiment, the peripheral device 20 may comprise an ambient sensor positioned in the environment of the user, such as a bed sensor, a smart mattress, a camera system (smartphone camera, infrared camera, thermal imaging camera, or connected home camera). Such sensor may detect presence/absence of movement, variations in respiratory rate, skin temperature changes, or visible signs of discomfort without requiring direct physical contact.

In yet another embodiment, the peripheral device 20 is a mobile device such as a smartphone or tablet, which itself integrates sensors (e.g., accelerometer, gyroscope, GPS, camera, microphone) and may be associated with external wearable or ambient sensors via wired and/or wireless communication protocols (e.g., Bluetooth®, Wi-Fi, ZigBee®). For instance, the camera of a mobile device may be used to perform remote photoplethysmography by detecting color variations in the skin corresponding to blood volume changes, thereby providing heart rate and SpO₂ measurements. Similarly, the microphone may capture breathing sounds to estimate respiratory rate, while inertial sensors may detect motion patterns or sudden falls.

In another embodiment, the peripheral device 20 may comprise medical-grade devices including, but not limited to, glucometers, continuous glucose monitors (CGMs) for real-time glucose tracking, pulse oximeters for measuring oxygen saturation (SpO₂) and pulse rate, ECG armbands or chest straps for continuous electrocardiogram monitoring, blood pressure cuffs (oscillometric or tonometric), digital thermometers or connected temperature patches, spirometers for lung function assessment, and medical-grade wearable patches incorporating combined sensors for instance to measure at least one of the Heart Rate, Respiratory Rate, and SpO₂.

For all the previous embodiments, the physiological sensor may include at least one of a photoplethysmography (PPG) sensor, an electrocardiography (ECG) electrode, an optical sensor, a pressure sensor, a temperature sensor, or a flow sensor.

The peripheral device 20 may also be configured to receive and/or acquire user-related information 23 beyond physiological parameters. Such information may be obtained using contextual sensors. For instance, such information may comprise the geographical location of the user (e.g., via GPS or triangulation of wireless signals), contextual activity data (e.g., walking, running, sleeping, or driving), environmental parameters (e.g., ambient temperature, humidity, air quality), or even behavioral indicators such as voice characteristics, facial expressions, or typing/motor patterns. These additional data provide valuable context for assessing the urgency of a medical situation.

The user-related information 23 may also be personal data entered by the user or his caregiver/doctor (i.e. received by the peripheral device 20 for instance via a user interface), which may include the height, weight, health risks, medical history, known allergies, ongoing treatments, prescribed medications, family medical background, lifestyle habits such as smoking or physical activity, and any physician-provided recommendations or thresholds to be taken into account for adapting the monitoring and analysis of the physiological signals 22.

In one embodiment, multiple physiological signals 22 and user-related information 23 are simultaneously acquired from multiple sensor sources.

The physiological signals 22 and user-related information 23 may be transmitted in the context of a remote monitoring session or a teleconsultation; submitted by a user through medical questionnaires or quality-of-life questionnaires; transmitted by connected medical devices. The present invention aims at solving a problem commonly faced by healthcare professional or institutions, that is the impossibility to fully appreciate, interpret and follow the evolution over time of the health states of a patient at a distant location, said health states comprise the patient's mental and emotional states; the symptoms and/or the side effects related to an illness or a treatment.

A technical problem addressed by the present invention is that healthcare professionals and institutions often lack means to reliably collect, synchronize, and interpret longitudinal health data from remote patients. The system enables continuous acquisition and remote access to physiological and contextual data, thereby supporting more accurate detection of evolving patient conditions, including symptoms or treatment-related side effects.

Furthermore, the present invention allows to store a user's data, such as the data collected by one or more connected objects; data obtained by means of machine learning algorithms; information submitted by a user, such as symptoms; physiological parameters, such as heart rates or other health indicators. Said user's data are analyzed while taking into account information submitted by the user, such as the answers to the questions of medical questionnaires relating to symptoms, to quality-of-life and to side effects and/or while taking into account information collected from the user, such as the duration of his connections, the searches he carried out, the communications between the user and the healthcare professionals. This analysis provides models aiming at improving medical research; understanding the evolution of a disease; and optimizing the treatment choice and prescription for each patient. Therefore, this system would allow to conduct real-life or real-world clinical studies, without time limit. In addition, the results of the user's data analysis can be used for building predictive models and/or for precision medicine.

It allows persistent storage of user data, such as measurements collected by connected devices, data derived from machine-learning algorithms, or information submitted by the user (e.g., symptoms, questionnaire responses). Physiological parameters, such as heart rate or oxygen saturation, may be combined with user inputs and contextual data to generate time-stamped records. The system analyzes these records by correlating multiple data sources, such as questionnaire responses with physiological trends or usage logs, to identify patterns indicative of disease evolution or treatment response. The analyzed data may further be used to build predictive models that improve the accuracy of medical risk detection and enable adaptive, individualized monitoring.

In the telehealth system according to the present invention, the questions of the questionnaires are provided by health professionals and/or are generated by means of algorithms. These algorithms that generate questions can do so on the basis of the parameters received from a user and/or they cross-reference said received parameters with other data, and generate questions based on the result of the cross-referencing. The cross-referencing may comprise: comparing different received parameters among them, comparing one or more received parameters with data stored in a database in the memory of the telehealth system, comparing one or more received parameters with user's answers to previous questions.

The questionnaires can differ from one patient to another depending on individual factors such as: health state, pathology, stage of a disease, treatment, age, sex or other individual factors.

In addition, the present system allows a patient to submit further questionnaires which he has answered to, such as pre-admission and/or post-admission questionnaires.

In what follows, the modules are to be understood as functional entities rather than material, physically distinct, components. They can consequently be embodied either as grouped together in a same tangible and concrete component, or distributed into several such components. Also, each of those modules is possibly itself shared between at least two physical components. In addition, the modules are implemented in hardware, software, firmware, or any mixed form thereof as well. They are preferably embodied within at least one processor of the telehealth system 100.

The telehealth system 100 may comprise a module 201 for receiving the at least two physiological signals 22 and the user-related information 23.

According to one embodiment, it is the peripheral device 20 of the telehealth system 100 that is configured to receive the at least two physiological signals 22 and the at least one user-related information 23.

For this purpose, the peripheral device 20 may comprise electronic parts such as an embedded acquisition circuit, an analog-to-digital converter (ADC), input/output interfaces, and communication units suitable for collecting and transferring physiological signals 22 and user-related information 23. The at least two physiological signals 22 and the user-related information 23 may be received continuously or intermittently from integrated sensors 21 or from external sensors 24 and transferred to the module 201.

When the sensor is integrated to the peripheral device 20 (e.g., integrated sensor 21), the signal may be acquired directly through an embedded circuit of the peripheral device 20 and transferred to the module 201 with minimal latency.

When the sensor is external to the peripheral device 20 (e.g., external sensor 24), the signal may be received through wired connections, such as Universal Serial Bus (USB) interfaces or standardized medical connectors, or through wireless communication protocols, including Bluetooth® Low Energy, Wi-Fi, ZigBee®, Near Field Communication (NFC), or cellular networks such as 4G or 5G.

In one embodiment, the received physiological signals 22 and/or user-related information 23 may first be acquired and temporarily stored in an internal volatile memory, such as Random Access Memory (RAM), of the peripheral device 20 to allow buffering and error correction. Alternatively or additionally, the received physiological signals 22 and/or user-related information 23 may be stored in a non-volatile memory, such as an Electrically Erasable Programmable Read-Only Memory (EEPROM) or flash storage, possibly within a Solid-State Disk (SSD), to ensure persistence in the event of power loss or transmission failure.

In embodiments where multiple physiological signals 22 and user-related information 23 are acquired simultaneously from multiple sources, the module 201 may assign timestamps and source identifiers to each incoming data stream. Furthermore, module 201 may synchronize signals arriving at different sampling rates by interpolating or resampling them to a common temporal reference, thereby allowing coherent multi-signal analysis and fusion in later stages.

The telehealth system 100 may optionally comprise a module 202 for preprocessing the received at least two physiological signals 22 and user-related information 23. Said preprocessing may include digital filtering to remove artefacts or noise, normalization of amplitude levels, detection and exclusion of corrupted or incomplete signals, and compression of data for optimized transmission.

Module 201 and module 202 may for instance be comprised in or be implemented by peripheral device 20 of the telehealth system 100. To that end, the peripheral device may comprise at least one processor configured to execute computer-executable instructions corresponding to the preprocessing operations. In some embodiments, a dedicated digital signal processor (DSP) or low-power microcontroller (MCU) may be used to perform real-time preprocessing locally, thereby reducing bandwidth consumption and ensuring that only relevant, processed data is uploaded.

Alternatively, the preprocessing may be performed at later stages, for instance by the central server 10 and the physiological signals 22 and user-related information 23 are thus directly transmitted to the central server 10 by the peripheral device 20.

The peripheral device 20 may thus comprise a module 203 (not represented) for transmitting the received (and optionally preprocessed) at least two physiological signals 22 and user-related information 23 to the central server 10.

Transmission may occur via secure short-range or long-range wireless communication protocols, including Bluetooth® Low Energy, Wi-Fi, or cellular networks such as 4G or 5G. Preferably, the communication is encrypted to ensure data privacy and integrity, with transmission latency reduced to less than one second in the case of 5G connectivity.

To that end, the peripheral device 20 may comprise one or more electronic components dedicated to communication, such as a wireless communication chipset or transceiver, an antenna module, and a communication controller operatively coupled to the peripheral device processor. The communication controller may manage protocol selection, error correction, and retransmission logic to ensure reliable data delivery. In some embodiments, the peripheral device 20 may further include a SIM card or embedded SIM (eSIM) for cellular connectivity, as well as secure hardware elements (e.g., Trusted Platform Module, TPM) for encryption key storage and authentication.

In embodiments involving multiple physiological signals 22 from heterogeneous sources, module 203 may prioritize transmission of critical medical parameters (e.g., abnormal heart rhythm, hypoxemia) while compressing or queuing less urgent data for delayed upload.

Transmission of critical medical parameters may be decided according to predefined medical thresholds. The predefined medical thresholds may notably be personalized to the patient condition, medical history, and evolving clinical profile. Such personalization may involve calibrating baseline values to the patient's usual physiological range (e.g., resting heart rate or SpO₂ levels), integrating known comorbidities (e.g., chronic obstructive pulmonary disease, diabetes, or cardiac insufficiency), and adapting to contextual factors such as age, medication, or recent clinical events.

For instance, parameters that cross urgency thresholds (e.g., arrhythmia detection, SpO₂ dropping below 90%, rapid blood pressure decline) may flagged as Tier 1 and transmitted with minimal latency. Less critical deviations (e.g., mild tachycardia, moderate desaturation) may be classified as Tier 2 and transmitted with intermediate priority, while routine background data (e.g., activity level, sleep quality, environmental conditions) may assigned to Tier 3 and compressed or queued for delayed upload.

The module 203 may further implement redundancy by selecting multiple available transmission paths to ensure robustness against network disruption. The outcome is that the central server 10 receives a coherent, synchronized, and medically relevant dataset representing the user's state in real time.

The telehealth system 100 may further comprise a module 204 for processing the received at least two physiological signals 22 and user-related information 23 so as to obtain a contextualized risk index. The contextualized risk index combines the physiological signals 22 and user-related information 23 into a unified and contextualized decision-making framework. The contextualized risk index is configured to increase the reliability and accuracy of the critical medical danger situation detection by interpreting the physiological signals 22 in context.

In one example, interpreting the physiological signals 22 in context may comprise comparing data related to the family relationships of a patient in order to draw conclusions on the possible appearance of symptoms. or pathologies from one family member to another, said data related to family relationships may be submitted by the patients themselves and/or by the healthcare professionals.

The contextualized risk index calculated by module 204 may be represented in various forms depending on the desired granularity of analysis. In one embodiment, the contextualized risk index is a scalar value, providing a single numerical score representing the overall likelihood of a critical medical danger situation. In alternative embodiments, the contextualized risk index may be represented as a vector, wherein each element corresponds to a contextualized risk index associated with a specific physiological parameter or category of user-related information. In further embodiments, the contextualized risk index may take the form of a matrix, encoding temporal or contextual information, with rows corresponding to individual physiological signals and columns representing discrete time windows, activity contexts, or environmental conditions, each element indicating a weighted contextualized risk index or probability of abnormality.

According to an embodiment, the processing operation to obtain the contextualized risk index may comprise at least one of a multi-sensor fusion, an adaptive weighting and a machine-learning based analysis.

Multi-sensor fusion refers to the integration of multiple physiological signals 22 from heterogeneous sensors, including the integrated sensors, external sensors and contextual sensors. Fusion may involve at least one of temporal alignment, normalization, correlation analysis, classification, feature extraction and a combination thereof.

In one example, correlation analysis may involve computing pairwise correlations (e.g., Pearson, Spearman), cross-correlations to detect lagged relationships, or multivariate correlations (e.g., principal component analysis, canonical correlation analysis) to evaluate dependencies across multiple physiological signals 22.

Feature extraction and combination may involve transforming the physiological signals 22 into comparable features (e.g., HRV indices, SpO₂ slopes, acceleration variance) or extracting specific features from the physiological signals 22 and aggregating them into a single feature vector for further analysis.

Different fusion techniques may be applied, including rule-based, probabilistic, or learned fusion approaches. Fusion may confirm the occurrence of an event by requiring coherent anomalies across multiple modalities (e.g., simultaneous immobility, SpO₂ drop, and abnormal heart rate variability), thereby improving detection reliability and reducing false positives. The fused result produces the contextualized risk index.

According to another embodiment, the contextualized risk index may be obtained using adaptive weighting. Each received physiological signal 22 may be associated with a dynamic weight that reflects its contextual reliability.

The weight attribution may for instance be carried out through at least one of:
a rule-based assignment (deterministic), using fixed medical rules (e.g., “if SpO₂ is inferior to 92% the weight is doubled relative to skin temperature”);
a statistical assignment, wherein a probabilistic model assigns weights based on statistical factors such as the variance and stability of each physiological signal 22. For instance, if a signal fluctuates wildly over a short period (high variance), it may indicate noise, artefacts, or poor sensor contact rather than a real physiological change. The weight is thus decreased. Conversely, a consistent signal remaining within expected physiological ranges can see its weight increased;
a user-personalized assignment, wherein weights are adjusted according to the user-related information 23 (e.g., user's health profile, age, chronic conditions, or medical history);

The weight attribution may further be contextually adjusted using the user-related information 23. For example, during detected immobility or a fall, SpO₂ and heart rate are given a higher weight, while during fever suspicion, temperature is given higher weight. Module 204 may then combine the weighted signals into the contextualized risk index.

According to yet another embodiment, the contextualized risk index may be obtained using a machine learning model configured to receive as input the physiological signals 22 and the user-related information 23 and to output a contextualized risk index. Supervised models such as Random Forest, Gradient Boosting, or Support Vector Machines (SVM) for classification tasks may be used. Alternatively, Neural Networks may be used, notably for pattern recognition when datasets are large (e.g., convolutional networks on ECG waveforms). Unsupervised models (e.g., clustering, autoencoders) may also be used for anomaly detection when labels are difficult to implement.

The machine learning model may be trained on datasets containing synchronized physiological signals, annotated events, and outcomes (e.g., hospital admissions, medical diagnoses). Data augmentation and cross-validation may be implemented to ensure generalization across different users and sensor noise conditions. In some embodiments, transfer learning may be applied, allowing personalization by fine-tuning a generic model with a small dataset specific to the user. The model may be repeatedly re-trained so that the model continuously adapts to the evolving baseline of each user, thereby reducing false positives and increasing detection robustness over time.

Module 204 may be implemented in the peripheral device 20, the central server 10, or distributed across both.

The peripheral device 20 may thus comprise a module 205 (not represented) for transmitting the contextualized risk index to the central server 10.

Therefore, according to a first embodiment, the peripheral device may implement module 201, module 203 and optionally module 202, while the central server 10 implements module 204 and optionally module 202.

According to a second embodiment, the peripheral device may implement module 201, module 204, module 205 and optionally module 202.

The telehealth system 100 may further comprise a module 206 for evaluating the contextualized risk index received from module 204 and determining whether a critical medical danger situation is occurring. According to an embodiment, module 206 is configured to apply a multi-layered comparison strategy. In other words, module 206 does not rely on a comparison with a single threshold, but instead compares the contextualized risk index against multiple families of reference data to ensure both robustness and safety. Notably, the contextualized risk index is compared to three families of references. The families of reference data are all active in parallel, but their contributions are hierarchized when synthesizing the decision.

A first family of reference data comprises general physiological references (i.e., established norms, thresholds and/or ranges, which serve as universal safety guardrails ensuring population-level safety). If values are outside of these bounds, it directly indicates a critical medical danger situation. General physiological references may be derived from external medical knowledge bases (e.g., guidelines, published ranges, validated clinical literature).

When the contextualized risk index is compared to the first family of reference data, the output is a flag or score indicating whether the contextualized risk index exceeds or falls below safe bounds, optionally together with the magnitude of deviation.

A second family of reference data comprises user-specific references. These user-specific references are personalized baselines derived from the historical data of the monitored user, such as median values, standard deviations, and contextual variations over defined time windows, allowing detection of intra-individual deviations even when measurements remain within normal ranges for the general population. For instance, the user-specific references may be directly derived from user-related information 23 received as input. They may be adjusted by healthcare professionals according to user characteristics (age, comorbidities, treatments), with traceability and configuration locking or be automatically adjusted based on the user's baseline and context (rest, activity, day/night).

When the contextualized risk index is compared to the second family of reference data, the output is a personalized deviation score, showing whether the current contextualized risk index represents an intra-individual anomaly, even if still within population norms. This quantifies the significance of deviation relative to the user's own physiology.

Finally, a third family of reference data comprises instrument-specific references (calibration state, noise level, such as sensor signal quality, motion artifacts, or battery status), which ensures that questionable measurements are invalidated or adjusted before contributing to the decision.

When the contextualized risk index is compared to the third family of reference data, the output is a signal reliability score or weight, indicating whether certain contextualized risk index values should be discounted, down-weighted, or invalidated because the sensor reading may be unreliable.

Comparisons of the contextualized risk index with the reference families may be performed over sliding temporal windows of configurable duration, for instance between 1 and 30 seconds, and may incorporate persistence controls such that transient or erratic variations do not directly trigger an alarm. The comparison process may further apply hysteresis functions, whereby the contextualized risk index must exceed or fall below a threshold by a defined margin or for a minimum duration before a deviation is considered significant, thereby preventing rapid oscillations or false triggers. Additionally, the process may include escalation delays, which define a temporal buffer before a detected deviation leads to a higher-level alert or intervention, ensuring that only persistent and coherent anomalies result in action.

According to one embodiment, in operation, instrument-specific references are applied first as a filter to validate the measurement quality. If the sensor signal is corrupted (e.g., motion artifacts, low SNR, battery failure), the data are either discarded, down-weighted, or flagged as unreliable before comparison with the other references. In this sense, the instrument-specific references help secure the reliability of the input. General physiological references and user-specific references (second level) may then be applied in parallel to the validated data. The outputs are then aggregated (e.g., weighted and combined) into a composite assessment.

Optionally, a machine learning model may further refine the evaluation of the contextualized risk index by detecting weak but consistent deviations across multiple parameters that may foreshadow an acute event. The model receives as input the contextualized risk index together with its underlying features (e.g., user baseline values, temporal signal patterns, and sensor reliability indicators), and analyzes their evolution over time to identify subtle but persistent shifts, such as a gradual increase in heart rate combined with a slight decrease in oxygen saturation. Based on these patterns, the model outputs a refined evaluation, for example a probability estimate or a severity classification, which serves as an early warning layer to anticipate critical medical danger situations before absolute thresholds are breached. The machine learning output complements, but does not override, the evaluation against general physiological reference data; any breach of general physiological reference threshold remains decisive.

Suitable machine learning models for refining the risk evaluation may include supervised classifiers such as Random Forests, Gradient Boosting Machines, Support Vector Machines, or neural networks. The choice of model may depend on practical constraints: lightweight models such as Random Forests or Support Vector Machines may be executed directly on the peripheral device (e.g., smartwatch or patch) where computational resources are limited, whereas more computationally intensive models such as deep neural networks or Gradient Boosting Machines may be executed on the central server where larger datasets and higher processing capacity are available. In either case, the model is applied to the contextualized risk index and its underlying features to produce a refined risk assessment in real time.

Training of the machine learning model may be performed on datasets comprising contextualized risk indexes and their underlying features, annotated with medical outcomes. Such datasets preferably contain both positive cases corresponding to acute medical events (e.g., hypoxemia, arrhythmia, syncope) and negative or control cases representing stable conditions. By training directly on contextualized risk indexes—already incorporating individual baselines and signal reliability indicators—the model learns to distinguish transient variations from clinically significant deterioration, thereby reducing false positives and improving robustness across heterogeneous users and devices. Standard techniques such as cross-validation and hyperparameter optimization may be applied to enhance generalizability. The trained model remains subject to the safeguard that breaches of general physiological reference thresholds automatically indicate a critical medical danger situation, ensuring that predictive refinements cannot compromise medically established safety thresholds.

According to a further embodiment, module 205 may implement a hybrid two-stage architecture comprising a deterministic stage and a machine-learning (ML) stage. The deterministic stage, which is mandatory uses the composite assessment trigger an alarm in case of critical events. The ML stage is optional and cannot override or suppress the deterministic stage.

For the deterministic stage, the system may apply layered criteria before triggering an alert. First, if the contextualized risk index breaches a general physiological reference threshold, an alert is triggered immediately. Otherwise, the system verifies that any deviation persists for a minimum duration within a sliding temporal window, and that at least two physiological parameters (e.g., SpO₂, heart rate, HRV) exhibit consistent anomalies. This combination ensures that transient noise does not cause false positives, while medically critical events are still detected without delay.

In a basic embodiment, the telehealth system 100 implements a binary classification at the output of module 206, indicating whether to initiate an emergency response or not initiate. In this context, “initiate” corresponds to sending an emergency message to at least one recipient device 31-35, whereas “not initiate” corresponds to continuing monitoring of the physiological signals and user-related information 23 without triggering any alert.

According to a more sophisticated embodiment, the binary “initiate” decision may be subdivided into multiple sub-categories reflecting the severity (i.e., level of criticality) and type of detected anomaly. The classification hierarchy can for instance include:
a critical level: if the contextualized risk index exceeds general physiological reference data, module 206 classifies the event as critical and triggers an emergency response by contacting professional emergency services directly (e.g., dialing 911). This sub-category represents the highest-priority alerts requiring immediate intervention.
a medium level: if the contextualized risk index indicates a significant deviation from the user's personalized baseline, or if the machine learning model identifies a high likelihood of a critical medical event, module 206 classifies the event as a medium level emergency. In this case, the system first alerts local responders, such as nearby family members, friends, or neighbors. If no acknowledgment or response is received within a predetermined time interval At, the system escalates the emergency message to professional emergency services.
a low level: if the anomaly is detected primarily through instrument-specific references, such as reduced signal quality, low SNR, or minor deviations in sensor readings, module 206 classifies the event as low-severity. The system may delay sending an emergency message but records the event for monitoring, logging, or future trend analysis, ensuring that persistent or escalating patterns are captured without unnecessarily alerting recipients.

In one embodiment, module 206 may be comprised in or be implemented by peripheral device 20 of the telehealth system 100. Alternatively, module 206 may be implemented at later stages, for instance by the central server 10.

The peripheral device 20 may thus comprise a module 207 (not represented) for transmitting the result of the contextualized risk index evaluation to the central server 10.

In practice, the result of the contextualized risk index evaluation is only transmitted when an emergency message must be sent. The contextualized risk index evaluation thus comprises the “initiate” binary output and optionally the level of severity of the detected critical medical danger situation.

Therefore, according to a first embodiment, the peripheral device may implement module 201, module 203 and optionally module 202, while the central server 10 implements module 204, module 206 and optionally module 202.

According to a second embodiment, the peripheral device may implement module 201, module 204, module 205 and optionally module 202, while the central server 10 implements module 206 and optionally module 202.

According to a third embodiment, the peripheral device may implement module 201, module 204, module 205, module 206 and optionally module 202.

The telehealth system 100 may comprise a module 208 for outputting an emergency message 25 based on the contextualized risk index evaluation. The central server 10 may be configured to send the emergency message 25 to at least one recipient device 31-35.

The emergency message 25 may comprise several elements. First, the emergency message 25 may include an alert signal indicating the existence of the critical medical danger situation. The emergency message 25 may further include additional information, such as the level of criticality, the type of detected anomaly, the position of the user, an entrance code or access instructions, the user's medical record, and any other contextual data useful to help rescue the user. The emergency message 25 may also include specific instructions on how to rescue the user, depending on the detected medical or situational emergency.

The emergency message 25 reception may lead to several outcomes: the study of a patient's case by a doctor or another healthcare professional; making a voice call, or launching a videoconference, to a patient's device by a doctor or another healthcare professional; automatically dispatching an ambulance, automatically dispatching a motorized convoy.

Said ambulance and/or motorized convoy may belong to an institution that utilizes the present telehealth system 100 or to one of its partners.

The dispatched ambulance service examines a patient and/or transports the patient by land, sea or air, for treatment or monitoring in a hospital, a clinic, a nursing home or other place of care delivery.

It has to be noted that the telehealth system 100 may allow prediction of different medical events, such as the evolution of a pathological condition known to the user, or the evolution of a novel pathological condition that may occur.

The system may further provide optional features supporting care coordination, such as: shared updating of the patient's medical record; educational content delivery; secure storage of administrative and medical documents; scheduling of teleconsultations or in-person consultations; secure messaging with healthcare professionals; a frequently asked questions area moderated by healthcare professionals; a conversational assistant for basic guidance; and a medical calendar aggregating upcoming care-related events.

The additional information may also comprise features related to the practical organization of the daily life of a patient. Said features comprise, for instance: the organization and update of the patient's medical record by various professionals with whom said medical record is shared; the personalized education of the patient about his/her one or more pathologies and treatments; the digitization of administrative and/or medical documents of the patient by means of a digital “safe” system; the scheduling of teleconsultations or in-person consultations with any healthcare professional; the secure message exchange with all the healthcare professionals, whether in a hospital or outside the hospital, such as ambulatory care facilities, that are in charge of said patient; a frequently asked questions forum moderated by healthcare professionals; a chatbot, or intelligent digital assistant based on machine learning, to answer patients' questions; and a medical calendar offering an overview of all the events in the healthcare path, such as blood tests, taken medications, or performed medical exams, said medical calendar being filled by the patient himself, by the healthcare professionals in charge of him, as well as automatically.

As a side note, the peripheral device 20 of the user may comprise a module configured to generate medication reminders on prescribed treatments and/or recorded conditions, said reminders being generated based on the patient's pathologies and/or the administered treatments or by means of an artificial intelligence based on prescribed treatments. The medication reminder module further allows visualization and validation by healthcare professionals, in order to avoid patient non-adherence. The reminder workflow may allow review and validation by healthcare professionals.

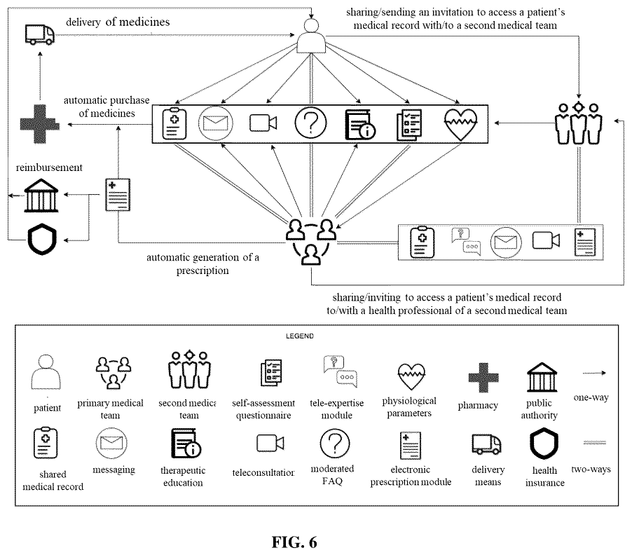

In some embodiments, the telehealth system 100 of the present invention may comprise features related to the practical organization of the daily life of healthcare professionals. On the health provider-side of the system (e.g., recipient devices 31-35), these features comprise a dashboard for displaying a summary of the tasks to be done or the tasks in progress; alerts related to a health state of the patients the health provider is in charge of, said alerts being customizable by means of criteria specific to the specialty of said health provider; recommendations related to a specific patient; automatically generated prescriptions; electronic medical records of the patients; a digital calendar; instant messages exchanged with their patients and/or colleagues by means of a secure, encryption-based, messaging module; an integrated teleconsultation module comprising a payment system, said teleconsultation module further comprising a speech recognition and recording module capable of generating a text report based on the recorded voice; a tele-expertise module in order to send requests to one or more colleagues; and a module for managing information concerning patients' illnesses or treatments. Advantageously, the digitalization and automation of multiple tasks performed by health professionals and the direct link between health professionals and colleagues and/or patients case the exchange of information and communication, thereby reducing workload and increasing productivity of health professionals. Examples of said links between health professionals and colleagues and/or patients according to one embodiment of the present invention are illustrated in FIG. 6.

In future embodiments, and only where permitted by applicable law and following clinical validation demonstrating non-inferiority or superiority to clinician review for specified indications, the prescription module may autonomously finalize prescriptions within predefined guardrails (e.g., formularies, dose ranges, contraindication and interaction checks), with full auditability, automatic pharmacovigilance reporting, and immediate human-override capability.

These features on the healthcare professional side of the telehealth system 100 further comprise technical means such as a prescription module configured to automatically generate or suggest medical prescriptions mentioning treatments and appropriate doses thereof, said module being capable of generating prescriptions in different formats by means of algorithms based on the patient's pathologies and/or the administered treatments, the data from the patient's shared medical record, the results of questionnaires, and the data collected from connected objects.

This prescription module may also be available to pharmacies. On the pharmacy side, the prescription module notifies about the need to make a drug purchase order when a prescription is generated, or it automatically generates said purchase order. The module may notify of the need to prepare a drug purchase order when a prescription is generated, or, when authorized by applicable regulations and explicit provider or patient consent, may initiate generation of such a purchase order.

The telehealth system 100 is capable of interpreting written, digital or oral prescriptions thanks to an artificial intelligence which converts human writing or oral voice into a digital format, and it further allows physicians to make electronic prescriptions by inserting the prescription directly in digital form, in a dedicated space of the system.

The telehealth system 100 may interpret written, digital, or oral prescriptions thanks to an artificial intelligence module that converts handwritten or oral inputs into structured digital data. Physicians may also create fully electronic prescriptions by directly entering them into a dedicated digital interface. In future validated embodiments, and subject to applicable regulatory approval, the artificial intelligence module may autonomously generate and finalize prescriptions within predefined medical guardrails (e.g., formularies, maximum dose ranges, contraindication and interaction checks). Such autonomous prescriptions remain subject to full auditability, real-time pharmacovigilance reporting, and immediate human override, ensuring that AI-based prescription assistance evolves safely toward non-inferior or superior performance compared to manual clinician review.

Furthermore, the telehealth system 100 allows the reorganization of institutions delivering care, such as hospitals, clinics, nursing homes or doctors' offices, so that hospital services, especially emergency services, may plan the admissions and discharges of future hospitalizations in advance.

The telehealth system 100 can be interoperable with other medical platforms providing other functions in medical institutions in France, in particular the DMP, or internationally, such as Cerner or Epic in the United States. The telehealth system 100 can interoperate with external medical platforms via standardized healthcare interfaces (e.g., HL7® FHIR®, HL7 v2, DICOM) and APIs, including national or institutional systems (e.g., DMP in France) and commercial EHRs (e.g., Cerner®, Epic®).

The central server 10 may include electronic components such as at least one processor, volatile memory (RAM), non-volatile memory (flash storage or SSD), and network interface circuitry configured to enable communication over multiple transmission channels, including wired Ethernet, Wi-Fi, or cellular networks (4G/5G). The recipient devices 31-35, which may be smartphones, tablets, wearable devices, or dedicated alert receivers, may comprise electronic components such as a processor, volatile and non-volatile memory, wireless network interfaces (Wi-Fi, Bluetooth®, NFC, or cellular), and optionally audio output modules for automated voice alerts.

In operation, the module 208 may transmit the emergency message 25 via multiple parallel communication layers to ensure delivery. For instance, the emergency message 25 may be sent over a secure IP channel using TLS/SSL for push notifications or API integration, while simultaneously sending a priority SMS via the cellular network as a fallback. Additionally, an automated voice call may be generated to guarantee reception on basic telephones or in situations where data connectivity is unavailable.

In addition, the telehealth system 100 may comprise a secure sharing module allowing access to a patient's data, said module enabling the patient and one or more authorized healthcare professionals to share all the patient's data with another healthcare team in a hospital in France or abroad. The sharing module may further comprise automated translation capabilities. In some embodiments, notification latency targets are configured on the order of sub-second for IP-based notifications and a few seconds for SMS delivery, with each transmission confirmed via an acknowledgment mechanism.

The module 208 may further implement redundancy and escalation logic, ensuring that each emergency message 25 is sent via at least two parallel channels. If the push notification fails, an SMS may be automatically transmitted. If the SMS fails, the automated voice call may be initiated. This multi-layered, redundant transmission approach increases the likelihood that at least one channel successfully delivers the emergency message 25, even in cases of network congestion, local outage, or device-specific failures. The device may further comprise several layers of resilience, including integrated battery backup for portable devices such as smartwatches or wearable medical sensors, automatic switching between Wi-Fi and cellular networks (Edge/3G/4G/5G) depending on availability, automatic low-power consumption mode in case of weak battery, reserving energy exclusively for alert transmission, permanent self-testing to warn the user if an alert cannot be transmitted, configurable time-to-initiation for automated voice call with synthetic message including identity, GPS localization, and vital data triggered in less than five seconds, and a parallel secure IP transmission to the medical regulation server for integration into their systems. Family members or relatives already alerted may simultaneously receive a notification indicating that the situation has become critical, in order to coordinate their action with emergency services.

Within the telehealth system 100, two distinct categories of recipient devices 31-35 may be defined. A first category comprises potential assistance provider recipient devices, which correspond to devices belonging to relatives, neighbors, friends, or other non-professional contacts previously authorized by the user to intervene in case of emergency. A second category comprises professional emergency recipient devices, which correspond to devices operated by emergency medical services, hospitals, clinics, or official rescue organizations.

Depending on the nature of the emergency message 25, one type of recipient device may be prioritized over the other. For instance, at lower levels of criticality, the module 208 may prioritize sending the emergency message 25 to potential assistance provider recipient devices, while at higher levels of criticality, it may prioritize sending the emergency message 25 to professional emergency recipient devices, including placing calls or messages via the appropriate local emergency access number.

Among the potential assistance provider recipient devices, the choice of priority may depend on several factors.

A proximity factor evaluates the physical or network proximity of each device, using GPS geolocation, Wi-Fi triangulation, or Bluetooth detection, in order to prioritize responders closest to the user.

A technical availability factor verifies that the device is connected, has sufficient battery, and is not in airplane or low-power mode.

A relevance factor evaluates whether the potential assistance provider is medically or relationally suitable, for example if the recipient is a designated caregiver, a family member familiar with the medical record, or a trained healthcare professional.

The prioritization algorithm may apply a hybrid weighting combining distance, qualification, and historical response time.

Escalation is automatic: if no contact responds within a predefined interval At, the alert is escalated to professional emergency recipient devices. For the most critical cases, such as cardiac arrest, anaphylaxis, or loss of consciousness, the system may immediately initiate emergency messages 25 both to potential assistance providers and to emergency services to minimize the risk of a missed response.

The selected potential assistance provider recipient device may acknowledge receipt of the emergency message 25 by sending an explicit confirmation, such as pressing an “I intervene” button on a mobile application, answering a voice prompt, or replying to an automated SMS. All received acknowledgments may be logged and classified by the telehealth system 100, enabling the system to either stop the escalation process or continue depending on the response. Contact availability may be managed dynamically through several mechanisms, including real-time network presence detection (Wi-Fi, cellular, Bluetooth), voluntary status settings (available, busy, absent), learning based on historical responsiveness to alerts, and redundancy mechanisms ensuring that even unavailable contacts may still receive backup notifications. If no acknowledgment is received within the predetermined interval At, the telehealth system 100 may either contact other potential assistance providers or escalate directly to professional emergency services.

The telehealth system 100 may further comprise continuous monitoring of the user, automatically recalculating the contextualized risk index in real time. If further deterioration is detected while an emergency message 25 is still active, the system may immediately notify additional recipient devices 31-35 and directly contact emergency services through a high-priority channel, ensuring uninterrupted response.

With regard to data management, the telehealth system 100 may be designed to collect only the information strictly necessary to ensure the efficiency of the alerts. Such information may comprise minimal identity (name and relationship with the user), communication data (telephone number, email, secure messaging identifier), user-defined proximity parameters (address, common Wi-Fi network, voluntary geolocation), historical data of responses to alerts (reaction time, type of response, notification opened or not), and contextual data linked to each event (sensor signals at the time of triggering, alert level, detected anomaly, list of contacted recipients and delivery status). No medical data on potential assistance providers may be recorded; their role being limited to receiving and responding to the emergency message 25.

Confidentiality may be ensured by several protection layers, including explicit consent from each potential assistance provider before inclusion in the alert list, encryption of data in transit (TLS/SSL) and at rest (AES-256), strict separation of databases so that third-party information is stored separately from medical data, limitation of use exclusively to alert triggering, access and erasure rights for each contact, and systematic event logging for security and auditing purposes.

The telehealth system 100 may implement a multilayer security model configured to support compliance with applicable healthcare data-protection frameworks (e.g., HIPAA, GDPR, HDS in France), including encryption in transit (e.g., TLS 1.3 with Perfect Forward Secrecy), encryption at rest (e.g., AES-256 with key rotation), granular access control with strong authentication, timestamped journaling of operations, optional zero-knowledge encryption, and end-to-end secure communications between sensors, user devices, servers, and recipient devices 31-35. For critical alerts, origin-authentication mechanisms (e.g., digitally signed messages or carrier-supported secure channels) may be used, where supported, to enhance authenticity and integrity.

It may be observed that the operations by the modules 201, 202, 203, 204, 205, 206, 207 and 208 are not necessarily successive in time, and may overlap, proceed in parallel or alternate, in any appropriate manner.

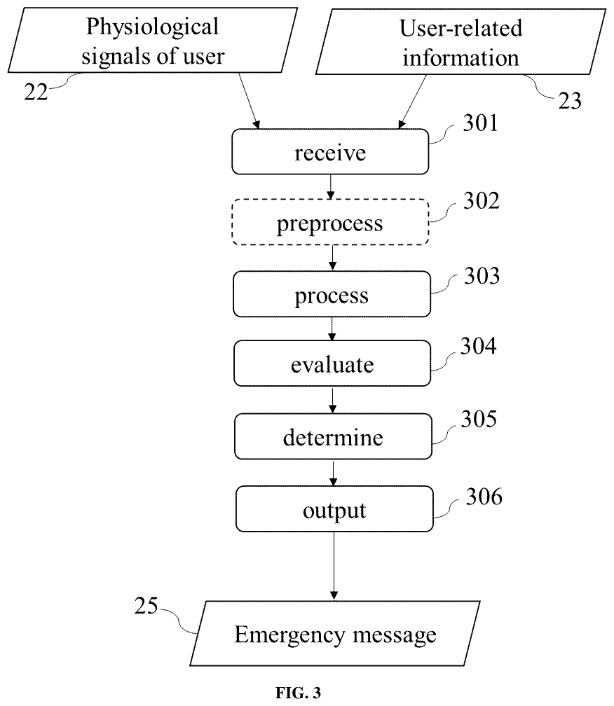

In its automatic actions, the telehealth system 100 may for example execute the following computer-implemented method 100 (FIG. 3):
receiving said at least two physiological signals and said at least one user-related information, said list of plurality of recipient devices and said plurality of reference data families, including physiological reference values, user-specific reference values, and instrument-specific reference values; (step 301)
optionally, preprocessing the data received as input; (step 302)
processing the received at least two physiological signals and user-related information into a contextualized risk index; (step 303)
evaluating the contextualized risk index against the plurality of reference data families, wherein the evaluation comprises a multi-level comparison including comparing he contextualized risk index to said general physiological reference data representing population-based medically established safety thresholds, wherein a breach of said general physiological reference data automatically indicates a critical medical danger situation regardless of other reference families; and refining the evaluation of the contextualized risk index using user-specific reference values representing individualized baselines, and instrument-specific reference values indicative of a reliability level of the acquired signals; (step 304)
determining, on the basis of said evaluation, whether said contextualized risk index indicates that the critical medical danger situation is occurring; (step 305)
outputting, when said critical medical danger situation is occurring, an emergency message to at least one recipient device from the list of the plurality of recipient devices, thereby triggering an intervention to rescue said user in response to the detected critical danger situation (step 306).

The central server 10, peripheral device 20 and recipient devices 30 may interact with a user interface 50, via which information can be entered and retrieved by a user. The user interface 50 includes any means appropriate for entering or retrieving data, information or instructions, notably visual, tactile and/or audio capacities that can encompass any or several of the following means as well known by a person skilled in the art: a screen, a keyboard, a trackball, a touchpad, a touchscreen, a loudspeaker, a voice recognition system.

A particular apparatus 9, visible on FIG. 5, is embodying at least one of the central server 10, the peripheral device 20 and the recipient devices 30 described above. It corresponds for example to a workstation, a laptop, a tablet, a smartphone, or a head-mounted display (HMD).

That apparatus 9 may comprise the following elements, connected to each other by a bus 95 of addresses and data that also transports a clock signal:
a microprocessor 91 (or CPU);
a graphics card 92 comprising several Graphical Processing Units (or GPUs) 920 and a Graphical Random Access Memory (GRAM) 921; the GPUs are quite suited to image processing, due to their highly parallel structure;
a non-volatile memory of ROM type 96;
a RAM 97;
one or several I/O (Input/Output) devices 94 such as for example a keyboard, a mouse, a trackball, a webcam; other modes for introduction of commands such as for example vocal recognition are also possible;
a power source 98; and
a radiofrequency unit 99.

According to a variant, the power supply 98 is external to the apparatus 9.

The apparatus 9 also comprises a display device 93 of display screen type directly connected to the graphics card 92 to display synthesized images calculated and composed in the graphics card. The use of a dedicated bus to connect the display device 93 to the graphics card 92 offers the advantage of having much greater data transmission bitrates and thus reducing the latency time for the displaying of images composed by the graphics card. According to a variant, a display device is external to apparatus 9 and is connected thereto by a cable or wirelessly for transmitting the display signals. The apparatus 9, for example through the graphics card 92, comprises an interface for transmission or connection adapted to transmit a display signal to an external display means such as for example an LCD or plasma screen or a video-projector. In this respect, the RF unit 99 can be used for wireless transmissions.

It is noted that the word “register” used hereinafter in the description of memories 97 and 921 can designate in each of the memories mentioned, a memory zone of low capacity (some binary data) as well as a memory zone of large capacity (enabling a whole program to be stored or all or part of the data representative of data calculated or to be displayed). Also, the registers represented for the RAM 97 and the GRAM 921 can be arranged and constituted in any manner, and each of them does not necessarily correspond to adjacent memory locations and can be distributed otherwise (which covers notably the situation in which one register includes several smaller registers).

When switched-on, the microprocessor 91 loads and executes the instructions of the program contained in the RAM 97.

As will be understood by a skilled person, the presence of the graphics card 92 is not mandatory, and can be replaced with entire CPU processing and/or simpler visualization implementations.

In variant modes, the apparatus 9 may include only the functionalities of the device 1, and not those of the device 2. In addition, the device 1 and/or the device 2 may be implemented differently than a standalone software, and an apparatus or set of apparatus comprising only parts of the apparatus 9 may be exploited through an API call or via a cloud interface.

1. A telehealth system for rescuing a user in a critical medical danger situation, the telehealth system comprising:
a central server, a plurality of recipient devices, and at least one peripheral device of the user;
the peripheral device and plurality of recipient devices being communicatively coupled to the central server; and
the peripheral device being configured to continuously or intermittently obtain at least two physiological signals indicative of a health condition of said user, and at least one user-related information;
wherein said telehealth system comprises:
at least one input configured to receive:
said at least two physiological signals and said at least one user-related information;
a list of the plurality of recipient devices; and
a plurality of reference data families, including physiological reference data, user-specific reference data, and instrument-specific reference data;
at least one processor configured to:
combine the received at least two physiological signals and user-related information into a contextualized risk index;
evaluate the contextualized risk index against the plurality of reference data families, wherein the evaluation comprises a multi-level comparison including:
comparing the contextualized risk index to said general physiological reference data representing population-based medically established safety thresholds, wherein a breach of said general physiological reference data automatically indicates a critical medical danger situation regardless of other reference data families; and
refining the evaluation of the contextualized risk index using user-specific reference values representing individualized baselines, and instrument-specific reference values indicative of a reliability level of the acquired signals; and
determine, on the basis of said evaluation, whether said contextualized risk index indicates that said critical medical danger situation is occurring;
at least one output configured to, when said critical medical danger situation is occurring, send an emergency message to at least one recipient device selected from the list of said plurality of recipient devices, thereby triggering an intervention to rescue said user in response to the detected critical medical danger situation.

2. The telehealth system from claim 1, wherein the evaluation of the contextualized risk index is performed over at least one sliding temporal window of configurable duration, and wherein a deviation of the contextualized risk index from at least one of said reference data families is considered for the evaluation of the contextualized risk index only if the deviation persists for a minimum duration within said sliding temporal window.

3. The telehealth system from claim 1, wherein the evaluation of the contextualized risk index further comprises applying a machine learning model configured to receive said contextualized risk index and to output a refined evaluation of the contextualized risk index, wherein the machine learning output does not override a deviation of the contextualized risk index from said general physiological reference data.

4. The telehealth system from claim 1, wherein the determination whether said critical medical danger situation is occurring comprises subdividing the critical medical danger situation into at least two levels of criticality, each level of criticality corresponding to a different severity of deviation of the contextualized risk index.

5. The telehealth system from claim 1, wherein the plurality of recipient devices comprises at least one potential assistance provider recipient device and at least one professional emergency recipient device.

6. The telehealth system from claim 4, wherein the plurality of recipient devices comprises at least one potential assistance provider recipient device and at least one professional emergency recipient device, and wherein said at least two levels of criticality include at least one lower level and at least one higher level, wherein in the at least one lower level, the at least one output is configured to prioritize sending said emergency message to said potential assistance provider recipient device, and in the at least one higher level, the at least one output is configured to prioritize sending said emergency message to said professional emergency recipient device.

7. The telehealth system from claim 5, wherein said at least one output is configured to send an emergency message to at least one potential assistance provider recipient device selected from the list of the plurality of recipient devices, wherein the selection is based on a multi-factor evaluation that combines: (i) a physical proximity of the potential assistance provider recipient device to the user, or a network-based proximity to the user, (ii) a technical availability of the potential assistance provider recipient device, and (iii) a medical or relational relevance of a potential assistance provider associated with the potential assistance provider recipient device.

8. The telehealth system from claim 5, wherein said at least one output is configured to send an emergency message to at least one potential assistance provider recipient device selected from the list of the plurality of recipient devices, wherein the selection is based on an evaluation of a physical proximity and/or a network proximity of each potential assistance provider recipient device to prioritize a potential assistance provider recipient device that is closest to the user and/or that is capable of receiving the emergency message with a lowest communication latency.

9. The telehealth system from claim 5, wherein if the selected potential assistance provider recipient device does not acknowledge receipt of the emergency message within a predetermined response time, the at least one output is configured to send the emergency message to an alternative potential assistance provider recipient device selected from the list of the plurality of recipient devices.

10. The telehealth system from claim 9, wherein successive alternative recipient devices are sequentially notified until at least one potential assistance provider recipient device acknowledges the emergency message.

11. The telehealth system from claim 5, wherein said at least one output is configured to escalate the emergency message to said at least one professional emergency recipient device if all potential assistance provider recipient devices fail to respond within a predetermined duration.

12. The telehealth system from claim 5, wherein said at least one output is configured to automatically escalate the emergency message to said at least one professional emergency recipient device if no acknowledgment of a potential assistance provider recipient device is recorded within said predetermined interval of time Δt.

13. The telehealth system from claim 5, wherein said at least one output is further configured to escalate the emergency message to professional emergency recipient devices upon a change in the level of criticality of the critical medical danger situation after an initial emergency message has been sent to at least one of said potential assistance provider recipient devices.

14. A computer-implemented method for rescuing a user in a critical medical danger situation, the method being implemented by a telehealth system comprising:
a central server, a plurality of recipient devices, and at least one peripheral device of the user;
the peripheral device and plurality of recipient devices being communicatively coupled to the central server; and
the peripheral device being configured to continuously or intermittently obtain at least two physiological signals indicative of a health condition of said user, and at least one user-related information;
wherein said method comprises:
receiving:
said at least two physiological signals and said at least one user-related information;
a list of the plurality of recipient devices;
a plurality of reference data families, including physiological reference data, user-specific-reference data, and instrument-specific reference data;
combining the received at least two physiological signals and user-related information into a contextualized risk index;
evaluating the contextualized risk index against the plurality of reference data families, wherein the evaluation comprises a multi-level comparison including:
comparing he contextualized risk index to said general physiological reference data representing population-based medically established safety thresholds, wherein a breach of said general physiological reference data automatically indicates a critical medical danger situation regardless of other reference families; and
refining the evaluation of the contextualized risk index using user-specific reference values representing individualized baselines, and instrument-specific reference values indicative of a reliability level of the acquired signals;
determining, on the basis of said evaluation, whether said contextualized risk index indicates that the critical medical danger situation is occurring; and
automatically outputting, when said critical medical danger situation is occurring, an emergency message to at least one recipient device from the list of the plurality of recipient devices, thereby triggering an intervention to rescue said user in response to the detected critical danger situation.

15. A computer program product comprising instructions which, when the program is executed by a computer, cause the computer to carry out the method according to claim 14.

16. A non-transitory program storage device, readable by a computer, tangibly embodying a program of instructions executable by the computer to perform the method according to claim 14.
